(19) United States
(12) Patent Application Publication
(10) Pub. No.: US 2005/0096627 A1
(43) Pub. Date: May 5, 2005

(54) Fluid aspiration device

(76) Inventor: Mark E. Howard, San Diego, CA (US)

Correspondence Address: Rodney F. Brown, 3365 Baltimore Street, San Diego, CA, 92117, US

(21) Appl. No.: 10/700,155

(22) Filed: Nov. 3, 2003

(51) Int. Cl. A61M 31/00

(52) U.S. Cl. 604/500; 600/578; 604/118

(57) ABSTRACT

A fluid aspiration device is provided having a syringe, a disposal reservoir and a disposal fluid passageway. The syringe has a variable-volume fluid chamber and an aspiration fluid passageway between the fluid chamber and a region of the body containing a fluid for aspiration. An aspiration valve is positioned in the aspiration fluid passageway. The disposal fluid passageway is between the fluid chamber and disposal reservoir and has a disposal valve positioned therein. The fluid is aspirated by expanding the variable volume of the fluid chamber to draw the fluid through the open aspiration valve into the fluid chamber. The aspiration valve is closed and the variable volume of the fluid chamber is contracted to open the disposal valve and displace the fluid into the disposal reservoir. When aspiration is terminated, the fluid aspiration device is maintained intact and the intact device including the fluid is disposed.

### TECHNICAL FIELD

The present invention relates generally to fluid aspiration, and more particularly to a device for aspiration of fluid from the body, sanitary disposal of the fluid, and optional collection of a fluid sample.

### BACKGROUND OF THE INVENTION

Localized accumulation of excess bodily fluid in an internal region of the body frequently occurs as a result of injury, infection, surgical trauma, or some other type of damage or disorder in that internal region of the body. It is generally desirable from a medical treatment standpoint to remove such excess accumulated bodily fluid from the body to reduce swelling and pain and to promote healing. Aspiration is a procedure for removing excess accumulated bodily fluid, which employs suction to draw the bodily fluid from the body. Aspiration procedures are commonly performed on joints. Aspiration of a joint is more specifically termed arthrocentesis. A syringe is the instrument of choice for performing arthrocentesis due to its simplicity and effectiveness.

In practice, the health care provider first preps the patient then inserts the needle of the syringe into the afflicted joint. The plunger of the syringe is manually pulled backward, displacing the plunger within the barrel of the syringe to create a suction. The suction draws the accumulated bodily fluid from the joint into the barrel of the syringe. When the barrel is filled, the health care provider withdraws the needle from the patient and disposes the aspirated bodily fluid in a sanitary manner, for example, by emptying the aspirated bodily fluid from the syringe into a disposal reservoir. It is oftentimes also desirable to retain samples of the aspirated bodily fluid for future diagnostic purposes.

With the existence and increased awareness of many blood-borne pathogens, the handling, sampling and disposal of medical waste, such as aspirated bodily fluids, has become more strictly regulated and correspondingly more costly and more problematic. U.S. Pat. No. 5,038,938 teaches a reservoir for disposing bodily fluids resulting from an arthrocentesis procedure in a relatively safe and regulatory compliant manner.

The present invention recognizes a continuing need for arthrocentesis devices, and more generally fluid aspiration devices, which enable effective performance of aspiration procedures and which further provide a safe and effective means for handling, sampling, and disposing the resulting aspirated bodily fluids. Accordingly, it is an object of the present invention to provide a fluid aspiration device, which enables effective performance of an aspiration procedure. More particularly, it is an object of the present invention to provide a fluid aspiration device, which is a safe and effective means for handling the resulting aspirated bodily fluid. It is another object of the present invention to provide a fluid aspiration device, which is a safe and effective means for disposing of the resulting aspirated bodily fluid. It is still another object of the present invention to provide a fluid aspiration device, which is a safe and effective means for sampling the resulting aspirated bodily fluid.

These objects and others are accomplished in accordance with the invention described hereafter.

### SUMMARY OF THE INVENTION

The present invention is a fluid aspiration device. The fluid aspiration device includes a syringe which has an aspiration intake and a fluid chamber. An aspiration valve is positioned in an aspiration fluid passageway between the aspiration intake and the fluid chamber. The fluid aspiration device also preferably includes fluid-tight connections between the aspiration intake and the aspiration valve and between the aspiration valve and the fluid chamber.

The aspiration valve has an open aspiration position and a closed disposal position. The aspiration valve is preferably biased in the closed disposal position and transitions from the closed disposal position to the open aspiration position in response to fluid pressure directed from the aspiration intake to the fluid chamber.

The fluid aspiration device further includes a disposal reservoir, a disposal fluid passageway between the fluid chamber and the disposal reservoir, and a disposal valve positioned in the disposal fluid passageway. The fluid aspiration device also preferably includes a junction fitting, a reservoir connector, and fluid-tight connections between the disposal valve and the disposal reservoir. The disposal fluid passageway preferably comprises a disposal line, the junction fitting and the reservoir connector in series.

The disposal valve has an open disposal position and a closed aspiration position. The disposal valve is preferably biased in the closed aspiration position and transitions from the closed aspiration position to the open disposal position in response to fluid pressure directed from the fluid chamber to the disposal reservoir. The disposal and aspiration valves are preferably mounted in a valve assembly, which is included in the syringe, in series between the aspiration intake and the fluid chamber.

In accordance with a specific embodiment of the present fluid aspiration device, a sampling outlet, a sampling fluid passageway, and a sampling valve are additionally included. The sampling fluid passageway is between the disposal reservoir and the sampling outlet. The sampling fluid passageway preferably comprises a sampling line, the junction fitting and the reservoir connector in series. The fluid aspiration device also preferably includes fluid-tight connections between the disposal reservoir and the sampling outlet. The syringe, disposal reservoir, disposal fluid passageway, sampling outlet, and sampling fluid passageway are preferably in fluid isolation from the external environment of the fluid aspiration device.

The sampling valve is positioned in the sampling fluid passageway and has an open sampling position and a closed non-sampling position. The sampling valve prevents fluid communication between the disposal reservoir and the sampling outlet in the closed non-sampling position and enables fluid communication between the disposal reservoir and the sampling outlet in the open sampling position.

The present invention is also a method for aspirating a fluid from a region of a body of a patient. The method provides a fluid aspiration device. The fluid aspiration device includes a syringe, an aspiration valve, a disposal reservoir, a disposal fluid passageway, and a disposal valve. The syringe has an aspiration intake, a fluid chamber with a variable volume, and an aspiration fluid passageway between the fluid chamber and the region of the body containing the fluid. The aspiration valve is positioned in the aspiration fluid passageway. The aspiration valve has an open aspiration position and a closed disposal position and the aspiration valve is fluid pressure actuated. The disposal fluid passageway is between the fluid chamber and the disposal reservoir. The disposal valve is positioned in the disposal fluid passageway. The disposal valve has an open disposal position and a closed aspiration position and the disposal valve is fluid pressure actuated. The fluid aspiration device is preferably maintained in fluid isolation from an external environment of the fluid aspiration device during practice of the present aspiration method.

The method proceeds by inserting the aspiration intake into the region of the body. The variable volume of the fluid chamber is expanded to create a suction in the fluid chamber and the fluid is drawn in an aspiration direction from the region of the body through the aspiration passageway in response to the suction. The aspiration valve is transitioned to the open aspiration position in response to fluid pressure in the aspiration direction and the fluid is further drawn through the aspiration valve in the open aspiration position into the fluid chamber in response to the suction.

The aspiration valve is then transitioned to the closed disposal position and the variable volume of the fluid chamber is contracted to displace the fluid from the fluid chamber into the disposal passageway in a disposal direction. The disposal valve is transitioned to the open disposal position in response to fluid pressure in the disposal direction and the fluid is displaced through the disposal valve in the open disposal position into the disposal reservoir in response to the contraction. The disposal valve is preferably transitioned thereafter to the closed aspiration position.

The sum of the above-recited method steps following insertion of the aspiration intake comprise an operating cycle of the fluid aspiration device. The present aspiration method may additionally include repeating the operating cycle one or more times. The method may also further include removing the aspiration intake from the region of the body after completing displacement of the fluid into the disposal reservoir in one of the operating cycles. The fluid aspiration device is maintained intact thereafter with the fluid retained therein and the intact fluid aspiration device including the fluid is disposed.

In accordance with a specific embodiment of the present aspiration method, the provided a fluid aspiration device further includes a sampling outlet, a sampling fluid passageway, and a sampling valve. The sampling fluid passageway is between the disposal reservoir and the sampling outlet. The sampling valve is positioned in the sampling fluid passageway. The sampling valve has an open sampling position and a closed non-sampling position. The sampling valve prevents fluid communication between the disposal reservoir and the sampling outlet in the closed non-sampling position and enables fluid communication between the disposal reservoir and the sampling outlet in the open sampling position.

The method further comprises placing a sampling container in fluid communication with the sampling outlet. The sampling valve is transitioned from the closed non-sampling position to the open sampling position and the fluid is conveyed from the disposal reservoir to the sampling container.

The present invention will be further understood from the drawings and the following detailed description.

### BRIEF DESCRIPTION OF THE DRAWINGS

FIG. 1 is a perspective view of a fluid aspiration device of the present invention.

FIG. 2 is a cross sectional view of a syringe of the fluid aspiration device of FIG. 1 in a fluid withdrawal mode of operation.

FIG. 3 is a cross sectional view of the syringe of the fluid aspiration device of FIG. 1 in a fluid disposal mode of operation.

### DESCRIPTION OF PREFERRED EMBODIMENTS

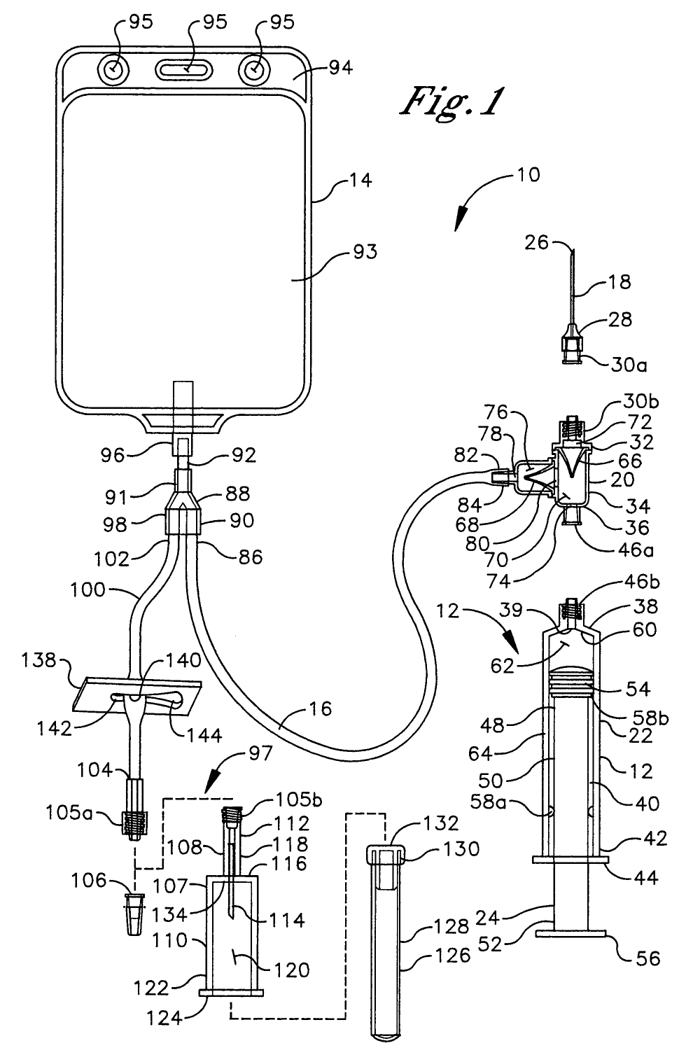

Referring to FIG. 1, an embodiment of a fluid aspiration device of the present invention is shown and generally designated 10. The fluid aspiration device 10 comprises a syringe 12, a disposal reservoir 14 and a disposal line 16 providing selective fluid communication between the syringe 12 and the disposal reservoir 14 in a manner described hereafter. The syringe 12 includes an aspiration intake 18, a valve assembly 20, a barrel 22 and a plunger 24, wherein the valve assembly 20 is positioned in series between the aspiration intake 18 and the barrel 22.

The aspiration intake 18 is preferably a conventional hollow syringe needle fabricated from a rigid metal and having an open front end 26, an open back end 28, and a continuous fluid passageway (not shown) extending therethrough. The front end 26 is a sharpened tip and the back end 28 is fixably attached in a fluid-tight manner to an aspiration intake coupler 30a, such as a releasable plastic male Luer lock fitting shown herein. The aspiration intake coupler 30a has a continuous fluid passageway extending therethrough, which is in fluid communication with the continuous fluid passageway of the aspiration intake 18 via the open back end 28 of the aspiration intake 18.

The valve assembly 20 has in series a front end 32, a mid-section 34, and a back end 36. An aspiration intake coupler 30b, such as a releasable plastic female Luer lock fitting shown herein, is also provided on the front end 32 of the valve assembly 20. The aspiration intake coupler 30b has a continuous fluid passageway extending therethrough into the valve assembly 20. The back end 28 of the aspiration intake 18 is releasably mounted to the front end 26 of the valve assembly 20 in a fluid-tight manner by means of the aspiration intake couplers 30a, 30b. The continuous fluid passageways of the aspiration intake couplers 30a, 30b are in fluid communication with one another as well as with the aspiration intake 18 and the valve assembly 20. Although not shown, it is within the scope of the present invention and the purview of the skilled artisan to substitute alternate conventional releasably connecting or fixably connecting aspiration intake couplers for the male and female Luer lock fittings 30a, 30b shown herein.

The barrel 22 has in series a front end 38 with a narrow opening 39 formed therein, a widened open interior 40, and an open back end 42. The back end 42 also has a barrel finger grip 44 integral therewith. A barrel coupler 46a, such as a releasable plastic female Luer lock fitting shown herein, is provided on the front end 38 of the barrel 22 which is in fluid communication with the interior 40 of the barrel 22 via the narrow opening 39 in the front end 38 of the barrel 22. The barrel coupler 46a has a continuous fluid passageway extending therethrough. A barrel coupler 46b, such as a releasable plastic male Luer lock fitting shown herein, is also provided on the back end 36 of the valve assembly 20. The barrel coupler 46b has a continuous fluid passageway extending therethrough into the valve assembly 20. The back end 36 of the valve assembly 20 is releasably mounted to the front end 38 of the barrel 22 in a fluid-tight manner by means of the barrel couplers 46a, 46b, wherein the continuous fluid passageways of the barrel couplers 46a, 46b are in fluid communication with one another as well as with the opening 39 and the valve assembly 20. Although not shown, it is within the scope of the present invention and the purview of the skilled artisan to substitute alternate conventional releasably connecting or fixably connecting barrel couplers for the male and female Luer lock fittings 46a, 46b shown herein.

The plunger 24 has in series an internal front end 48, a mid-section 50, and an external back end 52. The internal front end 48 has a plunger seal 54 mounted thereon and the external back end 52 has a plunger finger grip 56 integral with the plunger 24. A portion of the plunger 24, including the plunger seal 54, the internal front end 48 and a portion of the mid-section 50, is positioned at all times within the interior 40 of the barrel 22. The plunger 24 is slidably displaceable within the interior 40, but is not fully removable therefrom. Backward displacement of the plunger 24 within the interior 40 of the barrel 22 is limited by means of corresponding plunger stops 58a, 58b to prevent the plunger 24 from exiting the interior 40 of the barrel 22 via the open back end 42. The plunger stop 58a is mounted in the interior 40 of the barrel 22 proximal to the open back end 42 and engages the plunger stop 58b mounted on the internal front end 48 of the plunger 24 when the plunger 24 is withdrawn to its maximum extent from the interior 40 of the barrel 22 (i.e., maximum plunger withdrawal position) in a manner described hereafter. Forward displacement of the plunger 24 is limited by engagement of the plunger seal 54 with a front wall 60 of the barrel 22 when the plunger 24 is depressed to its maximum extent into the interior 40 of the barrel 22 (i.e., maximum plunger depression position) in a manner described hereafter.

Slidable displacement of the plunger 24 between the maximum plunger withdrawal and depression positions defines a variable-volume fluid chamber 62 within the interior 40 of the barrel 22. The variable-volume fluid chamber 62 is bounded by the plunger seal 54 at the internal front end 48 of the plunger 24, the front wall 60 of the barrel 22, and side walls 64 of the barrel 22. The actual volume of the variable-volume fluid chamber 62 ranges from essentially zero when the plunger 24 is at its maximum depression position (i.e., minimum depression volume) to approximately 20 cc, for example, when the plunger 24 is at its maximum withdrawal position (i.e., maximum withdrawal volume). A plurality of graduations (not shown) are preferably distributed along the length of the side walls 64 of the barrel 22 to enable the user to determine the volume of the variable-volume fluid chamber 62 at any position of the plunger 24. The user correlates the position of the plunger seal 54 with the specific adjacent graduation at a given time to fix the volume of the variable-volume fluid chamber 62 at that time.

The barrel 22 and plunger 24 are preferably fabricated from a disposable material, such as a relatively rigid or semi-rigid plastic. The barrel 22 is also preferably transparent or translucent. The plunger seal 54 is preferably fabricated from a relatively more pliant elastomeric material. The plunger seal 54 functions as a barrier to fluid flow from the variable-volume fluid chamber 62 past the plunger seal 54 into the interior 40 of the barrel 22 on the opposite side of the plunger seal 54 from the variable-volume fluid chamber 62.

The valve assembly 20 is a housing for an aspiration valve 66 and a disposal valve 68. The valve assembly 20 is preferably fabricated from a disposable material, such as a rigid plastic, and has an integral construction or is assembled from a plurality of separate components. The aspiration valve 66 is positioned in an aspiration valve chamber 70 having an aspiration inlet opening 72 and an aspiration outlet opening 74. The disposal valve 68 is correspondingly positioned in a disposal valve chamber 76 having a disposal outlet opening 78. The aspiration and disposal valve chambers 70, 76 share a common disposal opening 80 which is positioned between the aspiration valve chamber 70 and the disposal valve chamber 76 and provides fluid communication therebetween.

The aspiration inlet opening 72 is positioned adjacent to the aspiration intake coupler 30b and provides fluid communication between the continuous fluid passageway of the aspiration intake 18 and the aspiration valve chamber 70 via the back end 28 of the aspiration intake 18 and the continuous fluid passageways of the aspiration intake couplers 30a, 30b. The aspiration outlet opening 74 is positioned adjacent to the barrel coupler 46b and provides fluid communication between the aspiration valve chamber 70 and the variable-volume fluid chamber 62 via the narrow opening 39 at the front end 38 of the barrel 22 and the continuous fluid passageways of the barrel couplers 46a, 46b.

The valve assembly 20 is further provided with a disposal line coupler 82, which is shown herein as an integral nipple positioned on the mid-section 34 of the valve assembly 20. The disposal line coupler 82 is cross-sectionally sized in correspondence with the disposal line 16 for close-fitting fluid-tight insertion and retention in an open first end 84 of the disposal line 16. The disposal line coupler 82 has a continuous fluid passageway extending therethrough in fluid communication with the disposal outlet opening 78, which is positioned adjacent to the disposal line coupler 82. Accordingly, the disposal outlet opening 78 provides fluid communication between the disposal valve chamber 76 and the disposal reservoir 14 via the disposal line coupler 82 and the disposal line 16.

The aspiration valve 66 is a pressure-actuated one-way valve having a duckbill configuration. The aspiration valve 66 is biased in a closed disposal position with the paired duckbill flaps engaging one another as shown in FIG. 1 when no fluid pressure is applied to the aspiration valve 66. The aspiration valve 66 is transitioned to an open aspiration position with the paired duckbill flaps spread apart from one another as shown and described hereafter by applying a sufficient fluid pressure to the aspiration valve 66 in an aspiration direction (i.e., in a direction from the aspiration inlet opening 72 toward the aspiration and disposal valves 66, 68) to overcome the biasing force of the aspiration valve 66. The aspiration valve 66 returns to the closed disposal position when the fluid pressure to the aspiration valve 66 is either removed, decreased to a level below the biasing force of the aspiration valve 66, or applied in a disposal direction counter to the aspiration direction (i.e., in a direction from the aspiration outlet opening 74 toward the aspiration and disposal valves 66, 68).

The disposal valve 68 is similarly a pressure-actuated one-way valve having a duckbill configuration. The disposal valve 68 is biased in a closed aspiration position with the paired duckbill flaps engaging one another as shown in FIG. 1 when no fluid pressure is applied to the disposal valve 68. The disposal valve 68 is transitioned to an open disposal position with the paired duckbill flaps spread apart from one another as shown and described hereafter by applying a sufficient fluid pressure to the disposal valve 68 in the disposal direction to overcome the biasing force of the disposal valve 68. The disposal valve 68 returns to the closed aspiration position when the fluid pressure to the disposal valve 68 is either removed, decreased to a level below the biasing force of the disposal valve 68, or applied in the aspiration direction.

Although not shown, it is within the scope of the present invention and the purview of the skilled artisan to substitute alternate conventional valves for either or both of the one-way duckbill aspiration and disposal valves 66, 68 shown herein. The primary requirement of the alternate aspiration valve is that it permits flow in the aspiration direction, while preventing or substantially limiting flow in the disposal direction. Conversely, the primary requirement of the alternate disposal valve is that it permits flow in the disposal direction, while preventing or substantially limiting flow in the aspiration direction.

It is further within the scope of the present invention and the purview of the skilled artisan to modify the valve assembly 20 shown herein. For example, the aspiration and disposal valves 66, 68 may be repositioned in a single valve chamber of a modified valve assembly rather than in separate valve chambers 70, 76 of the valve assembly 20. Any number of other readily apparent structural modifications of the valve assembly 20, which do not obviate the desired function of the valve assembly 20 described below, are likewise within the scope of the present invention.

As recited above, the open first end 84 of the disposal line 16 receives the disposal line coupler 82 in fluid-tight engagement therewith. The disposal line 16 extends from the open first end 84 to an open second end 86 of the disposal line 16. An exemplary length of the disposal line 16 may be on the order of about 6 to 18 inches or more. The disposal line 16 is preferably fabricated from a clear flexible compressible plastic tubing having an inside diameter on the order of about ______. A continuous lumen (not shown) extends through the disposal line 16 from the first end 84 to the second end 86 of the disposal line 16, thereby providing a continuous fluid passageway between the first and second ends 84, 86.

A junction fitting 88 is provided downstream of the disposal line 16. The junction fitting 88 has an open disposal inlet port 90 and an open disposal outlet port 91, which are in fluid communication with one another via a common within the junction fitting 88. The second end 86 of the disposal line 16 is received into the disposal inlet port 90 and is attached in a fluid-tight manner thereto, providing a continuous fluid passageway between the disposal line 16 and the junction fitting 88. A relatively short reservoir connector 92 is received into the disposal outlet port 91 and is attached in a fluid-tight manner thereto. The reservoir connector 92 has a continuous fluid passageway extending therethrough, which is in fluid communication with the disposal outlet port 91. The reservoir connector 92 is preferably fabricated from the same or similar material as the material of the disposal line 16 and is cross-sectionally sized in correspondence with the disposal line 16 and the disposal outlet port 91.

The disposal reservoir 14 is a closed fluid-tight container having a volumetric capacity greater than the maximum withdrawal volume of the variable-volume fluid chamber 62 and preferably many times greater than the maximum withdrawal volume. An exemplary volumetric capacity of the disposal reservoir 14 is on the order of about 300 cc. The disposal reservoir 14 is preferably a flexible, fluid-tight, transparent or translucent, plastic bladder 93 and a peripheral tab 94 integral with the bladder 94, but in fluid isolation therefrom. The tab 94 has a plurality of cutouts 95 formed therein, which enable the practitioner to hang the disposal reservoir 14 from a hook or stand in the manner of an IV bag. A plurality of graduations (not shown) are preferably distributed along the height of the bladder 93 to enable the user to determine the volume of fluid in the disposal reservoir 14 at a given time. The disposal reservoir 14 is provided with a single open reservoir port 96, which is cross-sectionally sized in correspondence with the reservoir connector 92. The reservoir port 96 and the reservoir connector 92 are connected in a fluid-tight manner to provide a continuous fluid passageway between the reservoir connector 92 and the disposal reservoir 14.

The fluid aspiration device 10 may optionally include a sampling assembly generally designated 97 and described hereafter. In accordance with this embodiment as shown herein, the junction fitting 88 is configured as a “Y” fitting, which includes an open sampling port 98 in addition to the disposal inlet and outlet ports 90, 91. The sampling port 98 is in fluid communication with the disposal inlet and outlet ports 90, 91 via the junction fitting common. An open first end 102 of a relatively short sampling line 100 is received into the sampling port 98 and is attached in a fluid-tight manner thereto, providing a continuous fluid passageway between the sampling line 100 and the reservoir connector 92 via the disposal outlet port 91, junction fitting common, and sampling port 98. The sampling line 100 is preferably fabricated from the same tubing as the disposal line 16. As such, the sampling line 100 has a lumen which provides a continuous fluid passageway extending therethrough to an open second end 104 of the sampling line 100.

A sampling outlet coupler 105a, such as a releasable plastic female Luer lock fitting shown herein, is fixably attached in a fluid-tight manner to the open second end 104 of the sampling line 100. The sampling outlet coupler 105a has a continuous fluid passageway extending therethrough. A sampling outlet coupler plug 106, such as a releasable plastic male Luer lock plug shown herein, is provided, which is selectively releasably engagable with the sampling outlet coupler 105a. When the sampling outlet coupler plug 106 is placed in engagement with the sampling outlet coupler 105a, the sampling outlet coupler plug 106 prevents fluid flow out the sampling outlet coupler 105a via the open second end 104 of the sampling line 100.

The sampling assembly 97 further includes a sample transfer unit 107 comprising a sampling outlet 108 and a sampling guide 110. The sampling outlet 108 is a conventional hollow needle fabricated from a rigid metal and having an open front end 112, an open back end 114, and a continuous fluid passageway extending therethrough. The sampling guide 110 has in series a front end 116 with a narrow guide port 118 extending therefrom, a widened open interior 120, and an open back end 122. The back end 122 also has a retainer finger grip 124 integral with the sampling guide 110. The sampling guide 110 is preferably fabricated from the same or similar material as the barrel 22 and plunger 24.

The sampling outlet 108 is concentrically, fixably mounted in the guide port 118 of the sampling guide 110 in a fluid-tight manner such that no fluid flow is permitted between the inside wall of the guide port 118 and the outside wall of the sampling outlet 108. The open back end 114 of the sampling outlet 108 extends concentrically into the interior 120 of the sampling guide 110 and the open front end 112 of the sampling outlet 108 extends out of the sampling guide 110 in the opposite direction. An elastomeric self-resealing sampling outlet seal (not shown) in the form of a sheath is preferably positioned over the back end 114 of the sampling outlet 108 in the interior 120 of the sampling guide 110.

A sampling outlet coupler 105b, such as a releasable plastic male Luer lock fitting shown herein, is fixably attached to the end of the guide port 118 opposite the sampling guide 110. The sampling outlet coupler 105b has a continuous fluid passageway extending therethrough, in which the open front end 112 of the sampling outlet 108 is concentrically positioned. Accordingly, the back end 114 of the sampling outlet 108 is in fluid communication with the continuous fluid passageway of the sampling outlet coupler 105b via the open front end 112 and continuous passageway of the sampling outlet 108.

The sample transfer unit 107 is selectively releasably mountable to the sampling line 100 in a fluid-tight manner by means of the sampling outlet couplers 105a, 105b. When the sample transfer unit 107 is mounted to the sampling line 100, the continuous fluid passageways of the sampling outlet couplers 105a, 105b are in fluid communication with one another as well as with the sampling line 100 and the sampling outlet 108. Although not shown, it is within the scope of the present invention and the purview of the skilled artisan to substitute alternate conventional releasably connecting or fixably connecting sampling outlet couplers for the male and female Luer lock fittings 105a, 105b shown herein.

The back end 122 and interior 120 of the sampling guide 110 are preferably cross-sectionally sized to receive a sampling container 126. The sampling container 126 is preferably an evacuated glass test tube 128 sealed at its open end 130 with an elastomeric puncturable and self-resealing stopper 132. During a fluid sampling sequence, the sealed open end 130 of the sampling container 126 is inserted into the back end 122 of the sampling guide 110 until the stopper 132 engages a front wall 134 of the sampling guide 110. With the sampling container 126 so positioned, the sampling outlet seal is displaced to expose the open back end 114 of the sampling outlet 108, which punctures the stopper 132 and extends into the test tube 128 in a manner described hereafter.

A manual sampling valve 138 is provided in the sampling line 100. The sampling valve 138 is a rigid or semi-rigid plastic rectangular plate having an asymmetrical slot 140 cut therethrough. The slot 140 has a narrow segment 142 and a wide segment 144 at opposite ends of the slot 140. The sampling valve 138 is enabled by threading the sampling line 100 through the slot 140. In particular, when the sampling line 100 is manually slid into the narrow segment 142 of the slot 140, the sampling valve 138 is in a closed non-sampling position. As such, the width of the narrow segment 142 is sufficiently narrow that the side edges of the narrow segment 140 pinch the sampling line 100 closed, thereby preventing fluid flow through the sampling line 100 past the sampling valve 138 in either direction. Conversely, when the sampling line 100 is manually slid into the wide segment 144 of the slot 140, the sampling valve 138 is transitioned to an open sampling position. As such, the width of the wide segment 144 is sufficiently wide that the side edges of the wide segment 144 do not significantly crimp the sampling line 100, thereby permitting fluid flow through the sampling line 100 past the sampling valve 138 in either direction.

Although not shown, it is within the scope of the present invention and the purview of the skilled artisan to substitute alternate conventional valves for the manual sampling valve 138 shown herein. The primary requirement of the alternate valve is that it permits flow in a direction from the disposal reservoir 14 to the sampling outlet 108 when in the open sampling position and prevents flow in the direction from the disposal reservoir 14 to the sampling outlet 108 when in the closed non-sampling position.

It is understood that the sampling assembly 97, which includes components 98-144 of the fluid aspiration device 10 recited above, is optional insofar as it is within the scope of the present invention to omit the sampling assembly in its entirety from the fluid aspiration device of the present invention. The resulting fluid aspiration device having the sampling components omitted retains a fluid aspiration function, but lacks a fluid sampling function as described hereafter.

### Method of Operation

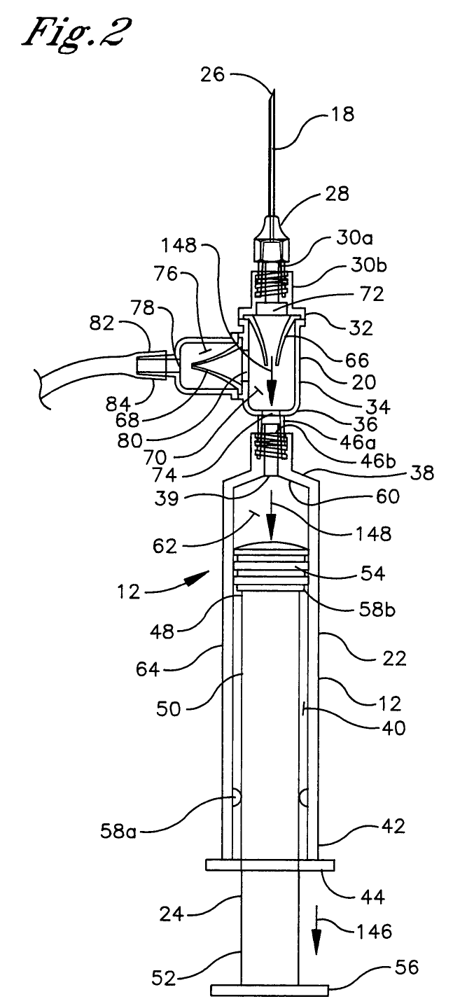

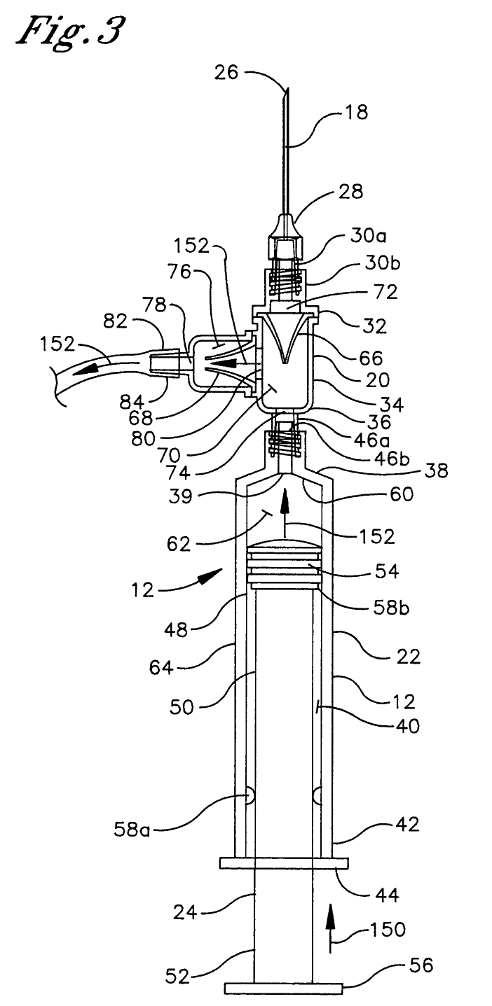

A method of operating the fluid aspiration device 10 is described hereafter with continuing reference to FIG. 1 and additional reference to FIGS. 2 and 3, wherein components in FIGS. 2 and 3, which are common to FIG. 1, are designated by the same reference character. The present operating method is generally characterized as a method for removing undesirable bodily fluid from an internal region of the body and subsequently disposing and/or sampling the removed fluid using the fluid aspiration device 10 described above. The operating method is specifically applicable to the removal of a fluid mixture of blood and joint fluid (specifically termed synovial fluid) from a joint, such as the knee, where the fluid mixture has undesirably accumulated by reason of injury, infection, surgical trauma, or some other type of joint damage or disorder. The fluid aspiration device 10 is used in accordance with this specific operating embodiment as an arthrocentesis device.

A number of preparatory steps are preferably performed before initiating the present operating method. In particular, the patient is preferably prepped by cleaning and sterilizing the skin external to a selected internal region of the body (not shown) from which the bodily fluid is to be removed. Patient prepping may also include injection of a local anesthesia into the selected internal region of the body. Preparatory steps further include ensuring that all fluid-tight connections for the components of the fluid aspiration device 10 are properly secured, that the plunger 24 is at the maximum plunger depression position in the barrel 22 of the syringe 12, that the sampling valve 138 is in the closed non-sampling position, and that the sampling outlet coupler plug 106 is placed in engagement with the sampling outlet coupler 105a. The practitioner then inserts the sharpened front end 26 of the aspiration intake 18 through the skin into the selected internal region of the body and ensures that the aspiration intake 18 is securely in place.

Referring initially to FIGS. 1 and 2, the present operating method commences with an aspiration sequence, wherein the practitioner grasps the barrel finger grip 44 and plunger finger grip 56 in each hand and manually pulls backward in the aspiration direction on the plunger finger grip 56 integral with the external back end 52 of the plunger 24 as shown by the directional arrow 146 in FIG. 2. Pulling backward in the aspiration direction on the plunger finger grip 56 correspondingly slidably displaces the plunger seal 54 backward in the aspiration direction through the interior 40 of the barrel 22 and expands the variable-volume fluid chamber 62. Expansion of the variable-volume fluid chamber 62 creates a vacuum therein. The resulting suction force maintains the disposal valve 68 in the closed aspiration position. The suction force also draws the bodily fluid from the selected internal region of the body through an aspiration fluid passageway including the aspiration intake 18, aspiration intake couplers 30a, 30b, and aspiration inlet opening 72 into contact with the aspiration valve 66 within the aspiration valve chamber 70. The aspiration valve 66, which is initially biased in the closed disposal position, responds to the fluid pressure of the bodily fluid in the aspiration direction by transitioning to the open aspiration position. The open aspiration valve 66 enables the bodily fluid to continue through the aspiration fluid passageway further including the aspiration valve chamber 70, aspiration outlet opening 74, barrel couplers 46a, 46b, and narrow front end opening 49 of the barrel 22 into the variable-volume fluid chamber 62 as shown by the flow arrows 148.

The practitioner continues backward manual displacement of the plunger 24 in the aspiration direction through the interior 40 of the barrel 22 until the plunger stops 58a, 58b engage one another at the maximum plunger withdrawal position. The variable-volume fluid chamber 62 correspondingly reaches its maximum withdrawal volume and is filled to capacity with the bodily fluid removed from the selected internal region of the body. At this point the aspiration sequence of the present operating method is completed.

Referring to FIGS. 1 and 3, the operating method continues with the disposal sequence, wherein the practitioner grasps the barrel finger grip 44 and plunger finger grip 56 in each hand and manually pushes the plunger finger grip 56 forward in the disposal direction as shown by the directional arrow 150 in FIG. 3. Pushing the plunger finger grip 56 forward in the disposal direction correspondingly slidably displaces the plunger seal 54 forward in the disposal direction through the interior 40 of the barrel 22 and contracts the variable-volume fluid chamber 62. Contraction of the variable-volume fluid chamber 62 displaces a portion of bodily fluid residing therein, termed the aspirate, forward in the disposal direction from the variable-volume fluid chamber 62 through a disposal fluid passageway including the narrow front end opening 49, barrel couplers 46a, 46b, aspiration outlet opening 74, and aspiration valve chamber 70 into contact with the aspiration valve 66 within the aspiration valve chamber 70. The aspiration valve 66, which is biased back to the closed disposal position after completion of the aspiration sequence, remains in the closed disposal position during the disposal sequence in response to the fluid pressure of the bodily fluid being displaced in the disposal direction. The closed aspiration valve 66 diverts the bodily fluid through the common disposal opening 80 into the disposal valve chamber 76 where the bodily fluid contacts the disposal valve 68. The closed aspiration valve 66 also prevents backflow of the bodily fluid through the aspiration intake 18 into the selected internal region of the body during the disposal sequence and from the aspiration intake 18 even after termination of operation described below.

The disposal valve 68 is initially in the closed aspiration position, but responds to the fluid pressure of the bodily fluid in the disposal direction by transitioning to the open disposal position. The open disposal valve 68 enables the bodily fluid to continue through the disposal fluid passageway further including the disposal valve chamber 76, disposal outlet opening 78, disposal line coupler 82, first end 84 of the disposal line 16, and lumen of the disposal line 16 as shown by the flow arrows 152. Although not shown in FIG. 3, displacement of the bodily fluid continues from the lumen of the disposal line 16 through the disposal fluid passageway further including the second end 86 of the disposal line 16, disposal inlet port 90, junction fitting common, disposal outlet port 91, reservoir connector 92 and reservoir port 96 into the disposal reservoir 14.

The practitioner continues forward manual displacement of the plunger 24 in the disposal direction through the interior 40 of the barrel 22 until the plunger seal 54 engages the front wall 60 of the barrel 22 at the maximum plunger depression position. The variable-volume fluid chamber 62 correspondingly reaches its minimum depression volume and is essentially free of any bodily fluid. At this point the disposal sequence of the present operating method is completed. A first operating cycle of the present operating method is likewise completed, which consists of the first aspiration sequence followed in series by the first disposal sequence. One or more additional operating cycles may be performed repetitively thereafter. It is noted that the disposal valve 68 is in the closed aspiration position during succeeding aspiration sequences in response to the suction force in the variable-volume fluid chamber 62 and the fluid pressure in the aspiration direction caused by the bodily fluid residing in the disposal reservoir and line 14, 16. The closed disposal valve 68 retains the bodily fluid in the disposal reservoir and line 14, 16 and prevents backflow of the bodily fluid into the syringe 12 during the aspiration sequences and even after termination of operation described below.

Operation of the fluid aspiration device 10 preferably continues until the disposal reservoir 14 is filled to capacity, all of the bodily fluid has been removed from the selected internal region of the body, or the practitioner no longer desires to remove additional bodily fluid from the selected internal region of the body. At this point operation of the fluid aspiration device 10 is terminated and the aspiration intake 18 is withdrawn from the selected internal region of the body out through the skin. It is apparent that the one-way valves 66, 68 and fluid-tight connections of the fluid aspiration device 10 prevent any undesirable leakage of bodily fluid from the device 10 during and after termination of operation. Accordingly, the intact fluid aspiration device 10 is in a condition for proper sanitary disposal in its entirety without any further handling of the device 10 upon termination of operation and withdrawal of the aspiration intake 18 from the patient. Sanitary disposal of the intact fluid aspiration device 10 correspondingly effectuates sanitary disposal of the bodily fluid residing therein.

A sampling sequence may optionally be performed at any time that sufficient bodily fluid resides in the fluid aspiration device 10 to reach the sampling container 126 positioned in the sampling guide 110. The sampling sequence is preferably performed when the disposal reservoir 14 is relatively full after termination of operation, but before disposal of the fluid aspiration device 10, or during operation of the fluid aspiration device 10, but preferably not during performance of an aspiration sequence. In accordance with the sampling sequence, the practitioner disengages the sampling outlet coupler plug 106 from the sampling outlet coupler 105a and connects the sampling outlet couplers 105a, 105b together. The practitioner then inserts the end 130 of the evacuated sampling container 126 into the interior 120 of the sampling guide 110 via the open back end 122. The practitioner displaces the sampling container 126 against the front wall 134 of the sampling guide 110, thereby pealing the sampling outlet seal away from the open back end 114 of the sampling outlet 108 and pushing the back end 114 through the stopper 132 into the evacuated test tube 128.

Once the sampling container 126 is securely in place in the sample transfer unit 107, the sampling valve 138 is manually transitioned from the closed non-sampling position to the open sampling position by manually sliding the sampling line 100 from the narrow segment 142 of the slot 140 in the sampling valve 138 into the wide segment 144 of the slot 140, thereby enabling fluid flow through the sampling line 100 past the sampling valve 138. Opening the sampling valve 138 provides fluid communication between the disposal reservoir 14 and the sampling container 126. Accordingly, the vacuum in the sampling container 126 draws a sample of bodily fluid from the disposal reservoir 14 through a sampling fluid passageway including the reservoir port 96, disposal outlet port 91, junction fitting common, sampling port 102, sampling line 100, sampling valve 138, sampling outlet couplers 105a, 105b, and sampling outlet 108 into the sampling container 126 when the sampling valve 138 is transitioned to the open position.

Once the sampling container 126 is filled with the sample of bodily fluid, the sampling valve 138 is transitioned back to the closed non-sampling position and the sampling container 126 is withdrawn from the sample transfer unit 107 via the open back end 122 of the sampling guide 110. Both the sampling outlet seal and the elastomeric stopper 132 are self-resealing once the sampling container 126 is removed from the sample transfer unit 107. The sampling outlet coupler plug 106 is also replaced in engagement with the sampling outlet coupler 105a. As a result, essentially no bodily fluid leaks from the fluid aspiration device 10 via the sampling assembly 97 at any time before, during or after the sampling sequence. In addition, the fluid-filled sampling container 126 can be stored or transported for subsequent analysis of the fluid sample without leakage of the sample from the sampling container 126.

An advantageous feature of the fluid aspiration device 10 is that the device 10 enables aspiration, disposal and optional sampling of bodily fluids without ever requiring the practitioner to handle the bodily fluids outside of the fluid aspiration device 10 and to risk unsafe contact therewith. The fluid passageways of the fluid aspiration device 10 remain closed to the external environment of the device 10 at all times during and after operation of the device 10 to minimize or eliminate the possibility of any bodily fluids escaping into the external environment and posing a contamination risk. It is noted that the term “external environment of the device 10” as used herein is not intended to include the body of the patient to which the device 10 is applied.

While the forgoing preferred embodiments of the invention have been described and shown, it is understood that alternatives and modifications, such as those suggested and others, may be made thereto and fall within the scope of the invention.

1. A fluid aspiration device comprising:
a syringe having an aspiration intake and a fluid chamber;
an aspiration valve positioned in an aspiration fluid passageway between said aspiration intake and said fluid chamber and having an open aspiration position and a closed disposal position, wherein said aspiration valve transitions from said closed disposal position to said open aspiration position in response to fluid pressure directed from said aspiration intake to said fluid chamber;
a disposal reservoir;
a disposal fluid passageway between said fluid chamber and said disposal reservoir; and
a disposal valve positioned in said disposal fluid passageway and having an open disposal position and a closed aspiration position, wherein said disposal valve transitions from said closed aspiration position to said open disposal position in response to fluid pressure directed from said fluid chamber to said disposal reservoir.

2. The fluid aspiration device of claim 1, wherein said aspiration valve and said disposal valve are mounted in a valve assembly included in said syringe in series between said aspiration intake and said fluid chamber.

3. The fluid aspiration device of claim 2, wherein said syringe, disposal reservoir, and disposal fluid passageway are in fluid isolation from the external environment of said fluid aspiration device.

4. The fluid aspiration device of claim 1 further comprising fluid-tight connections between said aspiration intake and said aspiration valve, said aspiration valve and said fluid chamber, and said disposal valve and said disposal reservoir.

5. The fluid aspiration device of claim 1, wherein said aspiration valve is biased in said closed disposal position.

6. The fluid aspiration device of claim 1, wherein said disposal valve is biased in said closed aspiration position.

7. The fluid aspiration device of claim 1, wherein said disposal fluid passageway comprises a flexible disposal tubing.

8. A fluid aspiration device comprising:
a syringe having an aspiration intake and a fluid chamber;
an aspiration valve positioned in an aspiration fluid passageway between said aspiration intake and said fluid chamber and having an open aspiration position and a closed disposal position, wherein said aspiration valve transitions from said closed disposal position to said open aspiration position in response to fluid pressure directed from said aspiration intake to said fluid chamber;
a disposal reservoir;
a disposal fluid passageway between said fluid chamber and said disposal reservoir;
a disposal valve positioned in said disposal fluid passageway and having an open disposal position and a closed aspiration position, wherein said disposal valve transitions from said closed aspiration position to said open disposal position in response to fluid pressure directed from said fluid chamber to said disposal reservoir;
a sampling outlet;
a sampling fluid passageway between said disposal reservoir and said sampling outlet; and
a sampling valve positioned in said sampling fluid passageway and having an open sampling position and a closed non-sampling position, wherein said sampling valve prevents fluid communication between said disposal reservoir and said sampling outlet in said closed non-sampling position and enables fluid communication between said disposal reservoir and said sampling outlet in said open sampling position.

9. The fluid aspiration device of claim 8, wherein said aspiration valve and said disposal valve are mounted in a valve assembly included in said syringe in series between said aspiration intake and said fluid chamber.

10. The fluid aspiration device of claim 9, wherein said syringe, disposal reservoir, disposal fluid passageway, sampling outlet, and sampling fluid passageway are in fluid isolation from the external environment of said fluid aspiration device.

11. The fluid aspiration device of claim 8 further comprising fluid-tight connections between said aspiration intake and said aspiration valve, said aspiration valve and said fluid chamber, said disposal valve and said disposal reservoir, and said disposal reservoir and said sampling outlet.

12. The fluid aspiration device of claim 8, wherein said aspiration valve is biased in said closed disposal position.

13. The fluid aspiration device of claim 8, wherein said disposal valve is biased in said closed aspiration position.

14. The fluid aspiration device of claim 8 further comprising a junction fitting and a reservoir connector, wherein said disposal fluid passageway comprises a disposal line, said junction fitting and said reservoir connector in series and said sampling fluid passageway comprises a sampling line, said junction fitting and said reservoir connector in series.

15. A method for aspirating a fluid from a region of a body of a patient comprising:
a) providing a fluid aspiration device including,
a syringe having an aspiration intake, a fluid chamber with a variable volume, and an aspiration fluid passageway between said region of said body containing said fluid and said fluid chamber,
an aspiration valve positioned in said aspiration fluid passageway, wherein said aspiration valve has an open aspiration position and a closed disposal position and said aspiration valve is fluid pressure actuated,
a disposal reservoir,
a disposal fluid passageway between said fluid chamber and said disposal reservoir, and
a disposal valve positioned in said disposal fluid passageway, wherein said disposal valve has an open disposal position and a closed aspiration position and said disposal valve is fluid pressure actuated;
b) inserting said aspiration intake into said region of said body;
expanding said variable volume of said fluid chamber to create a suction in said fluid chamber;
c) drawing said fluid in an aspiration direction from said region of said body through said aspiration passageway in response to said suction;
d) transitioning said aspiration valve to said open aspiration position in response to fluid pressure in said aspiration direction;
e) drawing said fluid through said aspiration valve in said open aspiration position into said fluid chamber in response to said suction;
f) transitioning said aspiration valve to said closed disposal position;
g) contracting said variable volume of said fluid chamber to displace said fluid from said fluid chamber into said disposal passageway in a disposal direction;
h) transitioning said disposal valve to said open disposal position in response to fluid pressure in said disposal direction; and
i) displacing said fluid through said disposal valve in said open disposal position into said disposal reservoir in response to said contraction, wherein steps c)-i) comprise an operating cycle of said fluid aspiration device.

16. The method of claim 15, wherein said operating cycle further comprises;
j) transitioning said disposal valve to said closed aspiration position.

17. The method of claim 15 further comprising repeating said operating cycle one or more times.

18. The method of claim 17 further comprising removing said aspiration intake from said region of said body after completion of step i) in one of said operating cycles, maintaining said fluid aspiration device intact with said fluid retained therein, and disposing said intact fluid aspiration device including said fluid.

19. The method of claim 15, wherein said fluid aspiration device is maintained in fluid isolation from an external environment of said fluid aspiration device.

20. The method of claim 15, wherein said fluid aspiration device further includes,
a sampling outlet,
a sampling fluid passageway between said disposal reservoir and said sampling outlet, and
a sampling valve positioned in said sampling fluid passageway and having an open sampling position and a closed non-sampling position, wherein said sampling valve prevents fluid communication between said disposal reservoir and said sampling outlet in said closed non-sampling position and enables fluid communication between said disposal reservoir and said sampling outlet in said open sampling position; and
said method further comprises;
placing a sampling container in fluid communication with said sampling outlet;
transitioning said sampling valve from said closed non-sampling position to said open sampling position; and
conveying said fluid from said disposal reservoir to said sampling container.
